(19) United States
(12) Patent Application Publication
(10) Pub. No.: US 2024/0164409 A1
(43) Pub. Date: May 23, 2024

(54) A METHOD OF REDUCING METHANE PRODUCTION IN A RUMINANT ANIMAL

(71) Applicant: DIT AgTech Limited, Wilsonton (AU)

(72) Inventors: VIVIENNE OLIVE MINTER MCCOLLUM, HAMPTON, QUEENSLAND (AU); MARK RAWLINSON PEART, GEHAM, QUEENSLAND (AU)

(21) Appl. No.: 18/282,623

(22) PCT Filed: Apr. 22, 2022
(86) PCT No.: PCT/AU22/50369

§ 371 Date: Sep. 18, 2023

(30) Foreign Application Priority Data
Apr. 23, 2021 (AU) 2021901206
Jul. 14, 2021 (AU) 2021902155

(51) Int. Cl. A23K 50/10; A23K 20/116; A23K 20/158; A23K 20/24

(52) U.S. Cl. CPC A23K 50/10; A23K 20/116; A23K 20/158; A23K 20/24

(57) ABSTRACT

A method of reducing methane production in a ruminant animal, comprising administering a methane reducer to a ruminant animal by proportionally dosing the methane reducer into a drinking water supply for the ruminant animal at a dosing rate selected to deliver the methane reducer to the ruminant animal in an effective manner.

### TECHNICAL FIELD

The present invention relates to a method of reducing methane production in a ruminant animal.

### BACKGROUND

Methane (CH₄) is a greenhouse gas produced primarily by methanogenic microbes that are found in natural ecosystems (e.g. wetlands, oceans and lakes) and the gastrointestinal tract of invertebrates and vertebrates, such as termites and ruminants. Methane is very effective in absorbing solar infrared radiation and has a global warming potential 25 times greater than CO₂. Consequently, its accumulation in the atmosphere contributes considerably to climate change. One of the main sources of anthropogenic CH₄ is ruminant livestock. In many countries cattle, sheep and goat production systems are largely pasture based and therefore attempts to reduce the carbon footprint must place considerable emphasis on methane emissions from extensively grazed ruminant animals with low quality forage diets.

Ruminants produce CH₄ as a by-product of the anaerobic microbial fermentation of feeds in the rumen and, to a lesser extent, in the large intestine. The ruminal microbial community is highly diverse and composed of bacteria, protozoa, fungi, and bacteriophages that act collectively to ferment ingested organic matter (OM) to produce short chain fatty acids that are absorbed across the rumen wall into the blood stream. However, anaerobic microbial fermentation in the rumen also produces carbon dioxide and hydrogen but if hydrogen is allowed to accumulate there is inhibition of both forage digestion and microbial growth. Methanogens such as Archaea present in the rumen use these end-products and produce CH₄. The production of CH₄ reduces the partial pressure of H₂ in the rumen, which could otherwise inhibit rumen fermentation, but it also reduces the amount of energy and carbon available for formation of the short chain fatty acids that are essential for ruminant nutrition. Furthermore, most of the CH₄ produced in ruminants is exhaled and belched by the animal and so increases atmospheric CH₄.

Mitigation strategies that reduce enteric CH₄ formation are important, and methods of reducing methane production in ruminant animals represent a major challenge, particularly for animals with low-quality forage diets. Mitigation strategies have been proposed which use feed additives that are classified (a) as methane inhibitors and act directly on the methanogenesis pathway or (b) as rumen modifiers that limit the growth of methanogens without specifically targeting the methanogenesis pathway (Honan, et al, 2021). Compounds that act as CH₄ inhibitors include 3-nitroxypropanol (3NOP), halogenated compounds such as bromoform and chloroform, and SUBSTITUTE SHEET (RULE 26) nitrates. As noted by Honan, et al (2021), more than 15 studies have been conducted using 3NOP, showing a marked reduction of enteric CH₄ emissions when 3NOP is introduced to ruminant diets as a feed additive. The macroalgae species Asparagopsis taxiformis and A. armata have been evaluated for their mitigation potential both in vitro and in vivo (Roque et al. 2019a, 2019b). Asparagopsis spp. contain relatively high concentrations of bromoform and other halogenated compounds such as bromochloromethane (Paul et al. 2006; Machado et al. 2016). Rumen modifiers include dietary lipids, medium chain fatty acids such as lauric, myristic, capric and caprylic acids, polyunsaturated fatty acids, probiotics, biochar, ionophores such as monensin, tannins, flavonoids, saponins, plant extracts and essential oils derived from cinnamon, lemongrass, ginger, garlic, juniper berries, eucalyptus, thyme, citrus, oregano, mint, rosemary and coriander, such as Agolin (Agolin, Bière, Switzerland; AR) which contains a blend of eugenol, geranyl acetate and coriander essential oils. Rasmussen and Harrison (2011) reported that the most effective fatty acid profiles that reduce CH₄ production were medium-chain (8-16 carbon chains (MCFA) and polyunsaturated (PUFA) fatty acids. However, there are concerns around animal welfare for CH₄ inhibitors and the consistency and effectiveness of rumen modifiers.

Beef production in northern Australia is on very large grazing properties with limited internal fencing with low levels of infrastructure and minimal labour input. Pasture management processes are difficult to implement. Large herds of Bos indicus and Bos indicus x Bos taurus cattle continuously graze tropical C4 pastures. Rainfall is highly seasonal. Nitrogen supplementation is undertaken during the extended dry season. The addition of non-protein nitrogen sources to low quality forage diets, typical of those consumed over the northern Australian dry season, increases forage intake and consequently live weight gain. The non-protein nitrogen source of choice is urea, and it is routinely delivered as a free-choice low-intake loose lick or lick block. It has been proposed (Callaghan et al, 2014) to replace urea with nitrate salts for the purposes of methane reduction; the reduction of nitrate to ammonia utilises hydrogen, diverting it from methanogenesis, and is more energetically favourable than methanogenesis.

However, there are challenges associated with nitrate supplementation. Nitrate compounds can be toxic to ruminants. Nitrate is reduced to nitrite by the rumen microflora and in some circumstances ruminal nitrite may increase to concentrations in excess of the conversion rate of nitrite to ammonia. In such circumstances blood nitrite concentrations may become sufficient to oxidise haemoglobin to methaemoglobin (MetHb). Methaemoglobin is unable to transport oxygen and hypoxia develops in the animal leading to dyspnoea and death. The diet of the animal can greatly affect nitrate toxicity, and the problem for northern Australia is that the type of highly digestible diets that would mitigate toxicity are not delivered by the forage base and supplementary feeding practices typical of northern Australia. Moreover, a single dose of nitrate is more toxic than the same amount of nitrate consumed over two or more intake events. In northern Australia supplementation is usually with loose licks or lick blocks. However, access to the lick blocks can result in competition between the animals, which can result in less dominant animals having restricted or no access to the lick blocks and dominant animals having access to lick blocks too often and for too long. Therefore, supplementation, with loose licks or lick blocks, as undertaken in northern Australia, increases the risk of nitrate toxicity.

The addition of encapsulated nitrate into ruminant diets was found to modulate the profiles of rumen archaea communities to lower methane production over time (Granja-Salcedo et al, 2019). Nitrates are effective methane inhibitors and a potential non-protein nitrogen source for cattle, acting as an H₂ sink and adding ammonia-based nitrogen to the rumen. In these experiments grazing animals were supplemented with concentrate composed of ground corn, soybean meal, mineral supplement and encapsulated nitrate (EN) supplement containing 70 g of EN/100 kg of BW, corresponding to 47 g NO₃/100 kg. The authors found that a solid, encapsulated nitrate is a feed additive that persistently affects enteric methane emissions. However, the additional cost of feeding an encapsulated nitrogen product means that this process is unlikely to be economic.

Accordingly, there remains a need for effective strategies for reducing methane production by ruminant animals.

### SUMMARY OF THE INVENTION

The present invention provides for a reduction in methane production in ruminant animals by introducing a methane reducer into a water supply for the ruminant animal. As a result, the present invention allows for a controlled and uniform supply of the methane reducer across each of the animals and thereby provides reduced methane production with a reduced risk of adverse effects on animal welfare, such as nitrate toxicity, and greater consistency in dosing.

Accordingly, in one aspect there is provided a method of reducing methane production in a ruminant animal, comprising administering a methane reducer to a ruminant animal by proportionally dosing the methane reducer into a drinking water supply for the ruminant animal at a dosing rate selected to deliver the methane reducer to the ruminant animal in an effective amount.

### DETAILED DESCRIPTION OF PREFERRED EMBODIMENTS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

In describing and claiming the present invention, the following terminology will be used in accordance with the definitions set out below. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, process or method that comprises a list of elements is not necessarily limited to only those elements but may include other elements not expressly listed or inherent to such composition, mixture, process or method.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” Thus, in some embodiments not otherwise explicitly recited, any instance of “comprising” may be replaced by “consisting of” or, alternatively, by “consisting essentially of”.

Also, the indefinite articles “a” and “an” preceding an element or component of the invention are intended to be non-restrictive regarding the number of instances (i.e., occurrences) of the element or component. Therefore “a” or “an” should be read to include one or at least one, and the singular word form of the element or component also includes the plural unless the number is obviously meant to be singular.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

As used herein, with reference to numbers in a range of numerals, the terms “about,” “approximately” and “substantially” are understood to refer to the range of −10% to +10% of the referenced number, preferably −5% to +5% of the referenced number, more preferably −1% to +1% of the referenced number, most preferably −0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

As used herein, wt % refers to the weight of a particular component relative to total weight of the referenced composition.

The term “and/or” used in the context of “X and/or Y” should be interpreted as “X,” or “Y,” or “X and Y.” Similarly, “at least one of X or Y” should be interpreted as “X,” or “Y,” or “both X and Y.”

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The present invention provides strategies for reducing methane production in ruminant animals which involve administering compounds that reduce methane production to ruminant animals in their drinking water rather than through dry feed supplementation such as lick blocks. This can be done by proportionally dosing a methane reducer into a drinking water supply for the ruminant animal, wherein the dosing rate is selected to provide the methane reducer to the ruminant animal in an effective amount.

It will be appreciated that introduction of a methane reducer into the water supply means the amount ingested by the animal will depend upon water intake, and the concentration of active ingredients and dosage rate are calculated to ensure administration of an appropriate amount. The daily water requirements and intake by livestock varies considerably according to class of stock, production status, age and condition of the animal, dry matter intake, quality and nature of feed, climatic conditions, and the quality of the water but this is well understood by the person skilled in art. For example, while the average daily water intake for beef cattle is about 45 L, in northern Australia hot summer temperatures significantly increase daily intake of water. Lactating cows may have a 30% higher daily water intake than dry cows. Furthermore, the requirements for Bos taurus cattle in hot conditions will be higher than those of Bos indicus cattle. Advantageously the methane reducer is proportionally dosed through dosing apparatus such as the uDOSE dosing units (DIT AgTech) so that dose rates may be adjusted to match herd characteristics and/or conditions.

The amount of water consumed by livestock animals is well understood. All ruminant animals will drink water proportional to their body weight each day. A dominant animal is unlikely to consume water in significantly greater quantities than a less dominant animal, therefore controlled and consistent administration of the methane reducer is achievable. Moreover, water intake can be monitored, such as with the uDOSE dosing unit, and controlled by controlling access to the water source.

As used herein, the term “methane reducer” refers to a substance that reduces methane production by a ruminant animal. The methane reducer may be a chemical compound or a composition including a mixture of chemical compounds. By way of example, a composition may be a blend, such as a blend of essential oils, or a composition containing one or more chemical compounds derived from an organism including plants, algae (including macroalgae) and microorganisms such as an extract from the organism. The substance may function as a methane inhibitor or as a rumen modifier.

In an embodiment, the methane reducer is water soluble. In this case it is administered dissolved in the drinking water. Alternatively, if substance is not water soluble it can be administered as a mixture with drinking water such as a dispersion or an emulsion. Suitable adjuvants such as emulsifying agents may be incorporated. Exemplary emulsifying agents include anionic emulsifying agents such as potassium laurate, triethanolamine stearate, sodium lauryl sulfate, alkyl polyoxyethylene sulfates, sodium dodecyl sulfate, and dioctyl sodium sulfosuccinate, nonionic surfactants such as polyoxyethylene fatty acid derivatives of the sorbitan esters (for example, Tween series), polyoxyethylene fatty alcohol ethers, sorbitan fatty acid esters, polyoxyethylene alkyl ethers (macrogols), polyoxyethylene sorbitan fatty acid esters, polyoxyethylene polyoxypropylene block copolymers (poloxamers), polyethylene glycol 400 monostearate, lanolin alcohols, and ethoxylated lanolin.

In an embodiment, the methane reducer is a methane inhibitor. A “methane inhibitor” is a substance that directly acts on the methanogenesis pathway in a way that can disrupt the process and reduce CH₄ production.

Methyl-coenzyme M reductase (MCR) is the enzyme that catalyses the final step of the methanogenesis pathway from an intermediate compound, methyl-CoM, to CH₄ and so inhibition of MCR inhibits methanogenesis and reduces methanogen growth.

In an embodiment, the methane inhibitor is an inhibitor of MCR. In an embodiment the MCR inhibitor is 3-nitrooxypropanol (3-NOP).

Halogenated compounds such as bromoform and chloroform have been found to interfere directly with the methanogenesis pathway by inhibiting a cobamide-dependent methyltransferase. Accordingly, in an embodiment the methane inhibitor is a cobamide-dependent methyltransferase inhibitor. In an embodiment, the cobamide-dependent methyltransferase inhibitor is a halogenated compound. In an embodiment, the cobamide-dependent methyltransferase inhibitor is a halohydrocarbon. In an embodiment, the cobamide-dependent methyltransferase inhibitor is a brominated hydrocarbon. In an embodiment, the cobamide-dependent methyltransferase inhibitor is bromoform. In an embodiment, the cobamide-dependent methyltransferase inhibitor is a chlorinated hydrocarbon. In an embodiment, the cobamide-dependent methyltransferase inhibitor is chloroform.

Organisms that accumulate halogenated compounds in their tissues have been investigated for their potential to reduce enteric CH₄ emissions. The macroalgae species Asparagopsis taxiformis and A. armata have been evaluated for their mitigation potential (Roque et al. 2019a, 2019b). Accordingly, in an embodiment the methane inhibitor comprises at least one species of red marine macroalgae applied in the form of a water-soluble extract therefrom or by administration of an active molecule derived therefrom such as bromoform. In an embodiment, the methane inhibitor comprises at least one red marine macroalgae of Asparagopsis species in the form of a water-soluble extract therefrom or an active molecule derived therefrom. In an embodiment, the species of Asparagopsis is A. taxiformis. In an embodiment, the species of Asparagopsis is A. armata. Solvent-based extraction techniques are well-known. Solvents used for the extraction of biomolecules from plants are chosen based on the polarity of the solute of interest. A solvent of similar polarity to the solute will properly dissolve the solute. Multiple solvents can be used sequentially in order to limit the amount of analogous compounds in the desired yield. The polarity, from least polar to most polar, of a few common solvents is as follows: Hexane<Chloroform<Ethyl acetate<Acetone<Methanol<Water. Extracts from Asparagopsis spp are described (Machado, et al 2016) and demonstrate that bromoform is the most abundant natural product in the biomass of Asparagopsis (1723 μg g⁻¹ dry weight [DW] biomass), followed by dibromochloromethane (15.8 μg g⁻¹ DW), bromochloroacetic acid (9.8 μg g⁻¹ DW) and dibromoacetic acid (0.9 μg g⁻¹ DW). Other methods, such as enzyme-assisted extraction, microwave-assisted extraction, pressurized liquid extraction, supercritical fluid extraction, and ultrasound-assisted extraction, which enable the extraction of biologically active compounds without their degradation, may be used.

In an embodiment, the methane reducer is a nitrate.

While not wishing to be bound by theory, it is believed that administration of a water soluble nitrate to a ruminant animal in drinking water provides an increase in non-protein nitrogen in the animal. Supplementation with non-protein nitrogen increases growth of the rumen microflora, which leads to more effective fibre utilisation and increased microbial protein production. Since the microbes are flushed out of the rumen in time and digested lower down the digestive system of the animal, the increase in non-protein nitrogen ultimately increases the availability of protein to the livestock animal. The present invention contemplates supplementing the diet of the ruminant animal with nitrate salts rather than conventional sources of non-protein nitrogen such as urea. While not wishing to be bound by theory, it is believed that microflora in the rumen undertake the reduction of nitrate to ammonia. This process utilises hydrogen, diverting it from methanogenesis, and is more energetically favourable than methanogenesis. Therefore, methane production is reduced. The expected methane reduction from supplying nitrate to a ruminant animal can be calculated by stoichiometry. During the reduction of nitrate to ammonia, 1 mole of nitrate (˜62 g) produces 1 mole of ammonia, which can be used as a nitrogen source by the animal and reduces methane production by 1 mole (˜16 g) (Callaghan et al, 2014).

The addition of non-protein nitrogen (NPN) increases forage intake and consequently liveweight gain under good conditions or, at least, reduces mortality and liveweight losses under difficult conditions such as those experienced in northern Australia during the dry season While not wishing to be bound by theory, the diversion of hydrogen from methanogenesis by the use of nitrate as a non-protein nitrogen source also reduces the non-productive consumption of carbon and this can contribute further to liveweight gain.

The present invention allows supplementation of the diet of a ruminant animal with a methane reducer with a reduced risk of harm to the animal since the dose is controlled. In an embodiment, the present invention allows administration of nitrate with reduced risk of nitrate toxicity. This can be done by proportionally dosing a solution of a water soluble nitrate into a drinking water supply for the ruminant animal, wherein the concentration of the solution and the dosing rate are selected to provide nitrate to the ruminant animal in a nutritionally effective amount that is below the level where nitrate toxicity is induced.

It will be appreciated that introduction of a water-soluble nitrate into the water supply means the amount ingested by the animal will depend upon water intake, and the concentration of active ingredients and dosage rate are calculated to ensure administration of an appropriate amount. The daily water requirements and intake by livestock varies considerably according to class of stock, production status, age and condition of the animal, dry matter intake, quality and nature of feed, climatic conditions, and the quality of the water but this is well understood by the person skilled in art. Advantageously the nitrate solution is proportionally dosed through dosing apparatus such as the uDOSE dosing units (DIT AgTech) so that dose rates may be adjusted to match herd characteristics and/or conditions.

The amount of water consumed by livestock animals is well understood. A dominant animal is unlikely to consume water in significantly greater quantities than a less dominant animal, therefore the prospect of consuming a toxic quantity of nitrate is reduced. Moreover, water intake can be monitored and controlled by controlling access to the water source. Therefore, a controlled and uniform supply of nitrate can be achieved.

Nitrate toxicity arises when nitrate is reduced to nitrite by the rumen microflora. In some circumstances ruminal nitrite may increase to concentrations in excess of the conversion rate of nitrite to ammonia. In such circumstances blood nitrite concentrations may become sufficient to oxidise haemoglobin to methaemoglobin (MetHb). Methaemoglobin is unable to transport oxygen and hypoxia develops in the animal leading to dyspnoea and death. The diet of the animal can greatly affect nitrate toxicity. Animals can be monitored for signs of nitrate poisoning. Symptoms of nitrate poisoning in domestic animals include increased heart rate and respiration; in advanced cases blood and tissue may turn a blue or brown colour. Water can be continuously monitored for nitrate concentration, or at least tested periodically.

Advantageously a dose less than 60 g/100 kg body weight is used. Preferably a dose less than 40 g/100 kg body weight is used when the type of highly digestible diets that would mitigate toxicity are not available. In an embodiment a dose of 10 g/100 kg body weight to 40 g/100 kg body weight is used is used. In an embodiment a dose of 20 g/100 kg body weight to 30 g/100 kg body weight is used is used. It will be appreciated that the person skilled in the art can select the concentration of methane reducer and the dosage rate to ensure administration of the methane reducer in the desired amount.

In an embodiment the dose of methane reducer starts at a lower level and increases. This addresses the possibility of an adaptive response to supplementation. For example, if supplementation is with nitrate, the possibility of an adaptive response in which nitrate reductase activity increases over time after feeding nitrate to an animal is reduced. More generally, it allows for the possibility of toxic effects to be observed and monitored at low levels before increasing towards to a level of methane reducer that may be closer to the toxic threshold for a herd.

As used herein the term “water soluble” or references to water solubility means that a chemical compound is capable of dissolving in water or a material that contains the element in question is capable of dissolving in water, more or less completely in an effective amount. In order to dissolve more or less completely there will be little or no solid residue in the water after a reasonable time has elapsed and where reasonable mixing steps have been undertaken. A compound is considered insoluble if its solubility is 0.1 mg/dL. Advantageously the methane reducer has a solubility of at least 1 mg/dL. In an embodiment, the methane reducer has a solubility of at least 5 mg/dL. In an embodiment, the methane reducer has a solubility of at least 10 mg/dL. In an embodiment, the methane reducer has a solubility of at least 50 mg/dL. In an embodiment, the methane reducer has a solubility of at least 100 mg/dL.

In an embodiment the nitrate is an inorganic nitrate salt. As will be well understood by the person skilled in the art, almost all inorganic nitrate salts are water soluble.

In an embodiment the nitrate is selected from the group consisting of aluminium nitrate, ammonium nitrate, barium nitrate, calcium nitrate, cerium(III) ammonium nitrate, cerium(III) nitrate, cerium(IV) ammonium nitrate, caesium nitrate, chromium(III) nitrate, cobalt(II) nitrate, copper(II) nitrate, iron(III) nitrate, magnesium nitrate, manganese(II) nitrate, nickel(II) nitrate, potassium nitrate, sodium nitrate and zinc nitrate, and hydrates thereof.

In an embodiment the nitrate is selected from the group consisting of ammonium nitrate, calcium nitrate, potassium nitrate and sodium nitrate.

In an embodiment the methane reducer is a rumen modifier. A “rumen modifier” as used herein is a substance that can modify the rumen environment to limit the growth of methanogens and/or suppress CH₄ production without targeting the methanogenesis pathway.

In an embodiment, the rumen modifier is selected from the group consisting of dietary lipids, medium chain fatty acids, polyunsaturated fatty acids, ionophores, tannins, flavonoids, saponins and essential oils.

Dietary lipids can modify the rumen environment as they have toxic characteristics for methanogens and protozoa. In addition, they can act as alternative hydrogen sink and increase the emphasis on propionate production, leading to reduction of enteric CH₄ production. Polyunsaturated fatty acids may also act as an alternative hydrogen sink as they may become hydrogenated within the rumen. Ionophores, such as monensin, alter rumen microbial populations to improve digestive efficiency by depriving methanogens of substrates that would otherwise be provided by microorganisms that have been reduced in number or eliminated by the ionophore. This shift favours the production of propionate over acetate, which reduces the amount of hydrogen available for methanogens.

In an embodiment the methane reducer is formulated as a physiologically acceptable composition comprising a physiologically acceptable carrier or diluent. A physiologically acceptable composition will usually comprise at least one adjuvant, diluent or carrier, which may be selected with due regard to the intended route of administration and standard practice in formulating supplements. Such carriers may be chemically inert to the active compounds and may have no detrimental side effects or toxicity under the conditions of use. The preparation of suitable formulations may be achieved routinely by the skilled person using routine techniques and/or in accordance with standard and/or accepted formulation practice.

In an embodiment the physiologically acceptable carrier or diluent is water.

In addition, the physiologically acceptable composition may comprise additives such as colouring agents, preservatives, surfactants and perfumes, as will be well understood by the person skilled in the art.

In an embodiment the physiologically acceptable composition may comprise further active ingredients. As used herein, the term “active ingredient”, or its equivalents, refers to substances that perform a role in enhancing the well-being of ruminant animals, as described herein. This may be by enhancing desirable process such as increasing non-protein nitrogen availability.

As used herein, the term “effective amount” refers to an amount that is sufficient to reduce methane production when introduced in that amount in the drinking water.

As used herein, the term “nutritionally effective amount” refers to an amount that will be effective in reducing methane production as well as enhancing a desirable process in an animal, such as increasing non-protein nitrogen availability when introduced in that amount in the drinking water. In the case of the water soluble nitrate, a nutritionally effective amount is an amount in the drinking water that is sufficient to reduce methane production and, at least in embodiments, to increase non-protein nitrogen intake in the animal.

In an embodiment the physiologically acceptable composition is formulated as a concentrate for dispensation into the water supply of ruminant animals. The concentrate can be administered by adding a measured amount to a source of drinking water such as a drinking trough. Advantageously the concentrate is proportionally dosed into a drinking water supply. In particular, it may be proportionally dosed through the uDOSE dosing units (DIT AgTech). In this case the dosing rate depends upon the concentration of the methane reducer in the concentrate and will be adjusted accordingly.

It is advantageous for the composition to be provided as a concentrated solution. Typically, the composition is provided in a container. Transport costs are minimised by transporting the least amount of water; hence it is advantageous for the composition to be concentrated. However, provision of a highly concentrated composition would generally require that the user dilute the composition. It has now been found that a concentrated composition can be proportionally dosed into the drinking water of a ruminant animal through a dosing unit such as the uDOSE dosing units (DIT AgTech). Accordingly, in an embodiment the composition is proportionally dosed into the drinking water of the ruminant animal directly from the container in which it is transported.

As used herein, the term “proportionally dosed” or its equivalents refers to a measured dispensation of a composition as described herein into a drinking water supply. The rate of dispensation is monitored and controlled to ensure that a desired concentration of the composition in the drinking water is achieved. This, in turn, ensures that a nutritionally effective amount of the active ingredients contained in the composition is delivered to animals drinking from the water supply. The rate of dispensation may be adjusted periodically to maintain the concentration of active ingredients in the drinking water supply if conditions change, or to adjust the concentration of active ingredients in the drinking water supply.

It will also be appreciated that administration of the methane reducer in very high amounts may not show enough benefit to justify the additional cost and can approach levels where toxicity, such as nitrate toxicity, could be induced. Adjustments can be made in the concentration of the methane reducer in the composition to be administered and/or in the rate of dispensing the composition so that the animal ingests an amount that is beneficial and cost effective. The reduction in methane production may be balanced against the economic cost. In embodiments where nitrate is the methane reducer, benefits to the animal of non-protein nitrogen supplementation are balanced against the risk. Accordingly, dosage is adjusted to ensure that nitrate poisoning, while mitigated against by the method of the present invention, does not occur. For example, in very hot weather, when more water is consumed, or if there are many lactating cows in a herd, the dose of nitrate may be reduced.

The person skilled in the art will understand that a user can monitor the beneficial effect of the non-protein supplementation by monitoring for signs such the weight of animals. In particular, they can compare the rate of weight gain (or reduction in weight loss in stressed animals) in animals treated with a nitrate and compare this to a baseline established for untreated animals. In addition, the person skilled in the art will understand that a user can monitor the reduction in methane by selecting animals from the herd and monitoring methane emissions from the selected animals over a period by capturing and measuring their emissions. Indirect calorimetry respiration chambers are often considered to be the ‘gold standard’ of methane measurement methods but involve large capital investment, are not ideally suited for use with large numbers of animals and require confinement of the animal, which may make such measurements not truly reflective of normal behaviour. However, non-dispersive infra-red (NDIR) sensor devices such as Guardian NG (Edinburgh Sensors) are capable of detecting methane production in cows in field environments and can be used to monitor a herd.

The present invention has application in reducing methane production in ruminant animals. Ruminant animals are polygastric, meaning their stomach is divided into compartments including the rumen. The rumen is adapted for the breakdown of fibre. It is the first stomach of a ruminant. The rumen receives food or cud from the oesophagus, partly digests it with the aid of bacteria, and passes it to the reticulum. Most ruminants belong to the family of bovids, Bovidae. The sub-family Bovinae, or bovines includes bison, buffalo, cattle, water buffalo, yak and zebu. The genus Ovis includes sheep. A third group of ruminants are the goat-antelopes, caprines of the sub-family Caprinae, which includes domestic and wild goats. A fourth group is the family Cervidae, which includes deer and elk. While the invention is applicable to all ruminant animals, it will be appreciated that it has most application to domestic species and, in particular, livestock animals. Therefore, in an embodiment the ruminant animal is selected from the group consisting of bison, buffalo, cattle, water buffalo, yak, zebu, sheep and goats.

### EXAMPLES

### Example 1

The impact of administering medication in drinking water as a delivery mechanism for supplementing beef cattle is compared to administration by incorporation in lick blocks. In the former, supplement is delivered in trough water, and thus supplement intakes are proportional to intakes of drinking water. This contrasts with conventional dosing by way of lick blocks and loose licks, which are driven by animal voluntary consumption. The recently developed uDOSE water medicator unit (DIT AgTech) uses mechanisms different to previous types of medicator units for regulating the amount of supplement delivered. These mechanisms include an electric diaphragm pump, a computer, and a nutrient meter to monitor dosing and automatically correct any mis-dosing if necessary. In the example responses of cattle medicated using a uDOSE unit are compared with medication with conventional lick blocks.

Cattle were grazed on a low soil phosphorus paddock. Soil tests conducted using the Colwell method indicated that most of the paddock (>95%) had soil phosphorus concentration of <2 mg/kg, while a small proportion of the paddock had phosphorus concentrations of 3-4 mg/kg.

To allow a direct comparison of supplementation methods, cattle in the study grazed the same paddock and were separated into two supplementation treatment groups by use of an auto-drafter. Trial cattle came through a ‘walk-over-weigh’ scales unit as they came into the water point with trough water. The cattle NILS tags were scanned through the walk-over-weigh unit and the animals were drafted accordingly into one of two ‘watering squares’ where the supplement was available. A total of 30 cows and 20 weaner steers were evenly allocated to two groups, i.e., to be supplemented with urea and phosphate by water medicator, or conventional lick blocks. All cows were pregnant at time of enrolment into the trial.

During the dry season, both groups were treated with a high urea content supplement using either uPro orange (DIT AgTech) in the water medicator group, or blocks containing 30% urea in the blocks group. During the wet season, both groups were on high P supplements using uPro green (DIT AgTech) in the medicator group or on blocks containing 8% phosphorus in the blocks group.

Consumption rates are based on adult equivalent (AE=450 kg dry animal), i.e., with AE as the denominator for consumption of water, urea, and P. Supplement consumption in the blocks group was calculated based on disappearance of the blocks, i.e., difference in weight, and urea/P content of the blocks. Medicator group supplement consumption was based on trough water consumption and dose rate of supplement into the water. Cow conceptus weight and lactation was not accounted for in the consumption/AE calculations.

Annual liveweight production was calculated as the change in cow weight over 12 months plus the weaner weight. Therefore, any cows not rearing a weaner had a weaner production of 0, and thus the liveweight production is a measure that takes pregnancy and calf survival into account. To determine weaner production on an individual cow basis, DNA testing was used to identify the dam of each calf.

This trial demonstrates that beef cattle fed via water medication technology have a higher intake of medication compared with animals fed via lick blocks. P consumption rates while cattle were on wet season supplement types were on average 8.2 g/AE·d in the medicator group and 0.3 g/AE·d in the blocks group (p<0.001)—a 2633% increase in intake. Urea consumption rates during the period cattle were fed the dry season supplement types were on average 56 g/AE·day in the medicator group, and 33 g/AE·d in the blocks group (p<0.05).—a 70% increase in intake. The higher levels of supplement delivery in medicator cows meant that they had an annual liveweight production (cow+weaner) 35 kg higher than the blocks group cows. Medicated steers had an annual liveweight production 22 kg greater than blocks group steers. Additionally, beef cows fed Phosphorus via water medication had higher levels of circulating inorganic P in their blood (serum) compared to those cows fed via lick blocks.

### Example 2

A range of feed additives have been demonstrated to directly suppress methane emissions from ruminants (e.g. Asparagopsis [Kinley et al., 2020], 3-NOP [Martinez-Fernandez et al., 2018], nitrate [Tomkins et al., 2018], essential oils [Roque et al., 2019c] and tannins [Yang et al., 2017]). These compounds suppress methane in experimental systems or when included in animal feed. While this is feasible in the intensive ruminant industries (dairy, feedlots) where controlled and sustained delivery to individual animals is possible, inclusion of these compounds in supplements of extensively grazed ruminants is problematic due to stability of compounds when exposed to the environment for extended periods and the sporadic and variable intake of supplements in extensive grazing scenarios (Dixon et al., 2003). As a result, adoption of many of these technologies and strategies may be a challenge in the extensive ruminant (cattle, sheep, goat) grazing systems used across Australia.

All ruminants in extensive grazing systems require a source of drinking water and, in the more extensive regions, this is increasingly delivered through artificial infrastructure (bore, tank, water trough) which can be monitored using sensor technology. A remotely managed direct water injection technology (DWIT), uDOSE (DIT AgTech), presents an opportunity to consistently deliver methane suppression compounds to ruminants grazing in extensive production systems of northern Australia. The use of the DWIT technology to safely deliver nitrates such as calcium nitrate, 3-NOP, bromoform, ionophores such as monensin and essential oils such as Agolin to ruminants via the drinking water and quantify the effect of the delivery mechanism on the efficacy of the compounds to suppress methane emissions is evaluated.

The effectiveness of these compounds in inhibiting methane production has been demonstrated once they have been introduced to the rumen. Solubility in water of selected methane reducers is as follows:

| Compound | Solubility in water | Characterised as |
|---|---|---|
| Calcium nitrate | 158.7 g/dL | Soluble |
| 3-nitrooxypropanol | | Soluble |
| (3NOP) | | |
| Bromoform | 3.2 g/L | Sparingly soluble |
| Monensin | | |
| Extract from | | Soluble |
| Asparagopsis | | |
| Agolin | | Soluble/dispersible |
| | | in water |

The compounds are mixed with water and are dosed using a uDOSE water medicator unit (DIT AgTech) into the drinking water supply for a mob of cattle as for the urea phosphate supplement of example 1. Bos indicus cattle are allocated to a paddock in a completely randomised design using an auto-drafter and walk-over-weigh (WOW) systems to allow exclusive access to treatments as in Example 1. Liveweight production, enteric methane emissions and changes in rumen fermentation characteristics and the rumen microbiome are measured.

Any reference to any prior art in this specification is not, and should not be taken as, an acknowledgement or any form of suggestion that the prior art forms part of the common general knowledge.

The complete disclosures of the patents, patent documents and publications cited herein are incorporated by reference in their entirety as if each were individually incorporated.

### REFERENCES

M. J. Callaghan, N. W. Tomkins, L. Benu and A. J. Parker. How feasible is it to replace urea with nitrates to mitigate greenhouse gas emissions from extensively managed beef cattle? Animal Production Science, 2014, 54, 1300-1304 http://dx.doi.org/10.1071/AN14270
Dixon, R M, White, A, Fry, P, Petherick, J C (2003) Effects of supplement type and previous experience on variability in intake of supplements by heifers. Australian Journal of Agricultural Research 54, 529-540.
Yury Tatiana Granja-Salcedo, Rodolfo Maciel Fernandes, Rafael Canonenco de Araujo, Luciano Takeshi Kishi, Telma Teresinha Berchielli, Flavio Dutra de Resende, Alexandre Berndt and Gustavo Rezende Siqueira. Long-Term Encapsulated Nitrate Supplementation Modulates Rumen Microbial Diversity and Rumen Fermentation to Reduce Methane Emission in Grazing Steers Front. Microbiol. https://doi.org/10.3389/fmicb.2019.00614
M. Honan, X. Feng, J. M. Tricarico and E. Kebreab. Feed additives as a strategic approach to reduce enteric methane production in cattle: modes of action, effectiveness and safety. Animal Production Science https://doi.org/10.1071/AN20295
Kinley R D, Martinez-Fernandez G, Matthews M K, de Nys R, Magnusson M, Tomkins N W (2020) Mitigating the carbon footprint and improving productivity of ruminant livestock agriculture using a red seaweed. Journal of Cleaner Production 259, 120836. doi:10.1016/j.jclepro.2020.120836
Machado, Lorenna, Magnusson, Marie, Paul, Nicholas A., Kinley, Robert, de Nys, Rocky, and Tomkins, Nigel (2016) Identification of bioactives from the red seaweed Asparagopsis taxiformis that promote antimethanogenic activity in vitro. Journal of Applied Phycology, 28 (5). pp. 3117-3126.
Martinez-Fernandez G, Duval S, Kindermann M, Schirra H J, Denman S E, McSweeney C S (2018) 3-NOP vs. halogenated compound: methane production, ruminal fermentation and microbial community response in forage fed cattle. Frontiers in Microbiology 9, 1582. doi:10.3389/fmicb.2018.01582
Paul N A, de Nys R, Steinberg P D (2006) Chemical defence against bacteria in the red alga Asparagopsis armata: linking structure with function. Marine Ecology Progress Series 306, 87-101. doi:10.3354/meps306087
Rasmussen J, Harrison A (2011) The benefits of supplementary fat in feed rations for ruminants with particular focus on reducing levels of methane production. ISRN Veterinary Science 2011, 613172. doi:10.5402/2011/613172
Roque B M, Brooke C G, Ladau J, Polley T, Marsh L J, Najafi N, Pandey P, Singh L, Kinley R, Salwen J K, Eloe-Fadrosh E (2019a) Effect of the macroalgae Asparagopsis taxiformis on methane production and rumen microbiome assemblage. Animal Microbiome 1, 3
Roque B M, Salwen J K, Kinley R, Kebreab E (2019b) Inclusion of Asparagopsis armata in lactating dairy cows' diet reduces enteric methane emission by over 50 percent. Journal of Cleaner Production 234, 132-138.
Roque B M, Van Lingen H J, Vrancken H, Kebreab E (2019c) Effect of Mootral: a garlic- and citrus-extract-based feed additive: on enteric methane emissions in feedlot cattle. Translational Animal Science 3, 1383-1388. doi:10.1093/tas/txz133
Yang K, Wei C, Zhao G Y, Xu Z W, Lin S X (2017) Effects of dietary supplementing tannic acid in the ration of beef cattle on rumen fermentation, methane emission, microbial flora and nutrient digestibility. Journal of Animal Physiology and Animal Nutrition 101, 302-310. doi:10.1111/jpn.12531

1. A method of reducing methane production in pasture-based ruminant animals, comprising administering a methane reducer to a ruminant animal by dispensing the methane reducer into a drinking water supply for the ruminant animal, wherein the rate of dispensation is monitored and controlled to ensure that a desired concentration of the methane reducer is maintained in the drinking water so as to deliver the methane reducer to the ruminant animal in an effective amount when the ruminant animal drinks.

2. A method as claimed in claim 1, wherein the methane reducer is a methane inhibitor.

3. A method as claimed in claim 2, wherein the methane inhibitor is an inhibitor of Methyl-coenzyme M reductase (MCR).

4. A method as claimed in claim 3, wherein the MCR inhibitor is 3-nitrooxypropanol (3-NOP).

5. A method as claimed in claim 2, wherein the methane inhibitor is a cobamide-dependent methyltransferase inhibitor.

6. A method as claimed in claim 5, wherein the cobamide-dependent methyltransferase inhibitor is a halogenated compound.

7. A method as claimed in claim 6, wherein the cobamide-dependent methyltransferase inhibitor is a brominated hydrocarbon.

8. A method as claimed in claim 7, wherein the cobamide-dependent methyltransferase inhibitor is bromoform.

9. A method as claimed in claim 5, wherein the cobamide-dependent methyltransferase inhibitor is a chlorinated hydrocarbon.

10. A method as claimed in claim 9, wherein the cobamide-dependent methyltransferase inhibitor is chloroform.

11. A method as claimed in claim 2, wherein the methane inhibitor comprises a water-soluble extract from at least one species of red marine macroalgae or an active compound derived therefrom.

12. A method as claimed in claim 11, wherein the at least one red marine macroalgae is of Asparagopsis species.

13. A method as claimed in claim 12, wherein the at least one red marine macroalgae is A. taxiformis.

14. A method as claimed in claim 12, wherein the at least one red marine macroalgae is A. armata.

15. A method as claimed in claim 2, wherein the methane inhibitor is a nitrate.

16. A method as claimed in claim 15, wherein the nitrate at least partially replaces urea as a non-protein nitrogen supplement.

17. A method as claimed in claim 15, wherein the water soluble nitrate is selected from the group consisting of aluminium nitrate, ammonium nitrate, barium nitrate, calcium nitrate, cerium(III) ammonium nitrate, cerium(III) nitrate, cerium(IV) ammonium nitrate, caesium nitrate, chromium(III) nitrate, cobalt(II) nitrate, copper(II) nitrate, iron(III) nitrate, magnesium nitrate, manganese(II) nitrate, nickel(II) nitrate, potassium nitrate, sodium nitrate and zinc nitrate, and hydrates thereof.

18. A method as claimed in claim 17, wherein the water soluble nitrate is selected from the group consisting of ammonium nitrate, calcium nitrate, potassium nitrate and sodium nitrate.

19. A method as claimed in claim 1 wherein the methane reducer is a rumen modifier.

20. A method as claimed in claim 19, wherein the rumen modifier is selected from the group consisting of dietary lipids, medium-chain fatty acids, polyunsaturated fatty acids, ionophores, tannins, flavonoids, saponins and essential oils.

21. A method as claimed in claim 1, wherein the ruminant animal grazes on tropical C4 pastures.

22. A method as claimed in claim 1, wherein the ruminant animal is selected from the group consisting of bison, buffalo, cattle, water buffalo, yak, zebu, sheep and goats.

23. A method as claimed in claim 1, wherein methane emissions are reduced.
